(19) United States
(12) Patent Application Publication
(10) Pub. No.: US 2007/0247786 A1
(43) Pub. Date: Oct. 25, 2007

(54) TORROIDAL BATTERY FOR USE IN IMPLANTABLE MEDICAL DEVICE

(76) Inventors: Paul B. Aamodt, Howard Lake, MN (US); Michael P. O'Brien, St. Anthony, MN (US)

Correspondence Address: MEDTRONIC, INC., 710 MEDTRONIC PARKWAY NE, MINNEAPOLIS, MN, 55432-9924, US

(21) Appl. No.: 11/379,977

(22) Filed: Apr. 24, 2006

(51) Int. Cl. H01G 9/08; H01G 2/10; A61N 1/00

(52) U.S. Cl. 361/517; 361/500; 607/116

(57) ABSTRACT

An implantable medical device is provided comprising a housing and circuitry disposed within the housing. A torroidal battery is disposed within the housing and coupled to the circuitry. The battery comprises a torroidal canister having a central opening therethrough and an electrode assembly disposed within the canister. An insulative body is disposed between the torroidal canister and the electrode assembly.

### TECHNICAL FIELD

This invention relates generally to an implantable medical device (IMD) and, more particularly, to a torroidal or doughnut-shaped battery for use within an IMD.

### BACKGROUND OF THE INVENTION

A wide variety of implantable medical devices (IMDs) exists today, including various types of pacemakers, cochlear implants, defibrillators, neurostimulators, and active drug pumps. Though IMDs may vary in function and design, many have common design features and goals. It is a common goal, for example, that every IMD should be made as compact as possible, without sacrificing device performance, so as to minimize the amount of trauma and/or discomfort that implantation of the device might cause a patient. Additionally, virtually every IMD must be provided with some type of power source, typically an electrochemical cell or battery that occupies a significant volume of space within the canister of the IMD. Consequently, the size of the battery may have a strong impact on the overall size and shape of the IMD. Moreover, the battery's capacity often determines how long an IMD may remain implanted in a patient without the need for servicing. In view of this, a primary goal in the production of IMDs is to minimize battery volume without causing a corresponding loss in capacity.

The battery of an IMD typically comprises a metal housing (e.g., titanium, aluminum, steel, etc.) having a cavity therein to accommodate an electrode assembly. The electrode assembly, which is electrically insulated from the housing by an insulative body (e.g., a polypropylene insert), may comprise an anode, a cathode, and one or more insulative separator sheets (e.g., a polymeric film) disposed intermediate the anode and cathode. Each electrode may include a lead or tab extending therefrom that may be electrically coupled (e.g., laser welded) to, for example, the canister of the IMD or circuitry disposed within the IMD. The canister is typically filled with an electrolytic fluid to provide a medium for ionic conduction between the anode and the cathode.

The configuration of the electrode assembly may vary by battery type. IMDs often employ spiral wound or cylindrical batteries, which utilize a coiled electrode assembly to increase the active surface area of the electrodes and maximize current carrying capacity. In such a battery, the electrodes and the separator take the form of long foil strips, which are wrapped around a mandrill having a relatively narrow outer diameter. The mandrill is then removed leaving a coiled electrode assembly having a generally cylindrical shape. The coiled electrode assembly is then placed into a cylindrical housing, which is filled with an electrolytic fluid and finally capped.

As stated above, cylindrical batteries are volumetrically efficient, largely due to their utilization of a coiled electrode assembly. However, cylindrical batteries do suffer from certain limitations. To minimize volume in a cylindrical battery, the central coils or innermost turns of the electrode assembly are made to be especially tight. This requirement for tight windings may lead to the delamination of the electrode mix (e.g., silver vanadium oxide) due to excessive bending of the current collector. Additionally, the electrode assembly may exhibit a spring-like resiliency and physically resist being so tightly coiled. If the assembly undergoes radial expansion after coiling, it may be difficult to insert the electrode assembly into the battery housing. To overcome such resiliency-related problems, a sizing process may be performed wherein the electrode assembly is placed under pressure to flatten the cylinder and to reduce assembly “spring-back”.

Considering the foregoing, it should be appreciated that it would be desirable to provide a battery suitable for use in an implantable medical device that occupies a reduced volume of space without having a diminished capacity. In addition, it would be advantageous if such a battery employed a coiled electrode assembly, but did not suffer from the limitations (e.g., active material delamination) associated with the cylindrical battery designs discussed above. Furthermore, other desirable features and characteristics of the present invention will become apparent from the subsequent detailed description of the invention and the appended claims, taken in conjunction with the accompanying drawings and this background of the invention.

### BRIEF DESCRIPTION OF THE DRAWINGS

The following drawings are illustrative of particular embodiments of the invention and therefore do not limit the scope of the invention, but are presented to assist in providing a proper understanding. The drawings are not to scale (unless so stated) and are intended for use in conjunction with the explanations in the following detailed descriptions. The present invention will hereinafter be described in conjunction with the appended drawings, wherein like reference numerals denote like elements, and:

FIG. 1 is an isometric view of a torroidal battery in accordance with a first embodiment of the present invention;

FIG. 2 is a isometric view of a shelf provided on the torroidal battery shown in FIG. 1;

FIG. 3 is a partially exploded view of the torroidal battery shown in FIG. 1;

FIG. 4 is an isometric view of the electrode assembly of the torroidal battery shown in FIGS. 1-3;

FIG. 5 is a top view of a shelf of the torroidal battery shown in FIGS. 1-3 illustrating the bonding of the electrode assembly;

FIG. 6 is an exploded view an implantable medical device;

FIG. 7 is an isometric cutaway view of a pulse generator employed in the implantable medical device shown in FIG. 6 incorporating the torroidal battery shown in FIGS. 1-3; and

FIG. 8 is an exploded view of a torroidal battery in accordance with a second embodiment of the present invention.

### DETAILED DESCRIPTION OF THE EXEMPLARY EMBODIMENT

The following description is exemplary in nature and is not intended to limit the scope, applicability, or configuration of the invention in any way. Rather, the following description provides a convenient illustration for implementing an exemplary embodiment of the invention. Various changes to the described embodiment may be made in the function and arrangement of the elements described herein without departing from the scope of the invention.

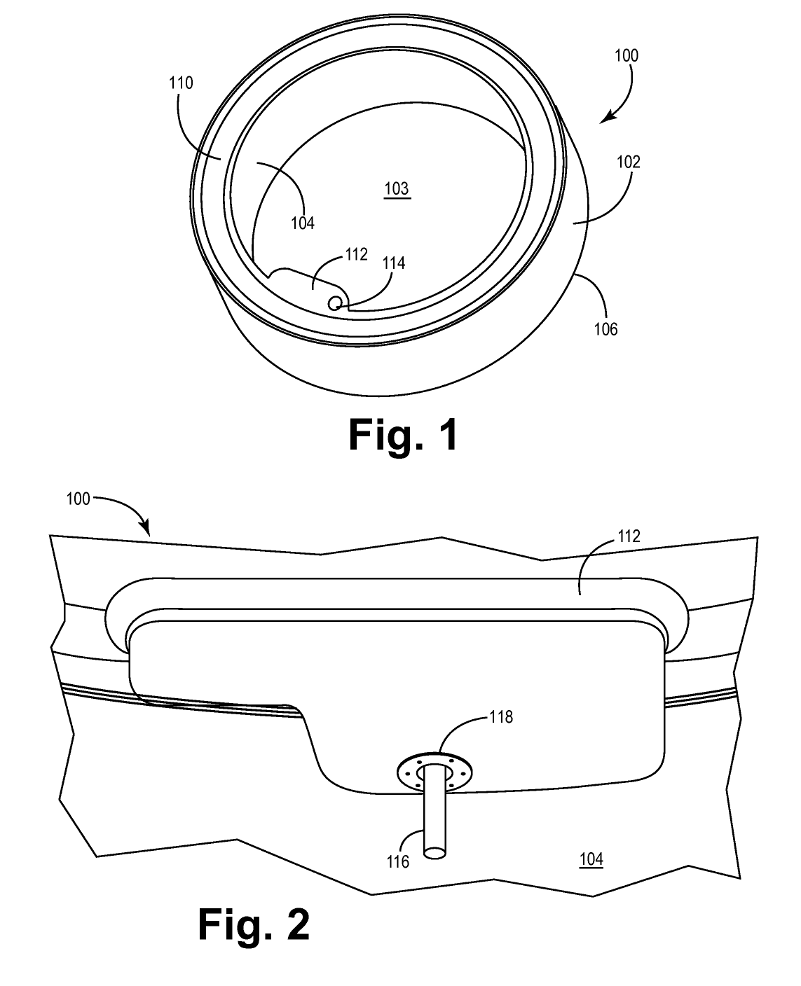

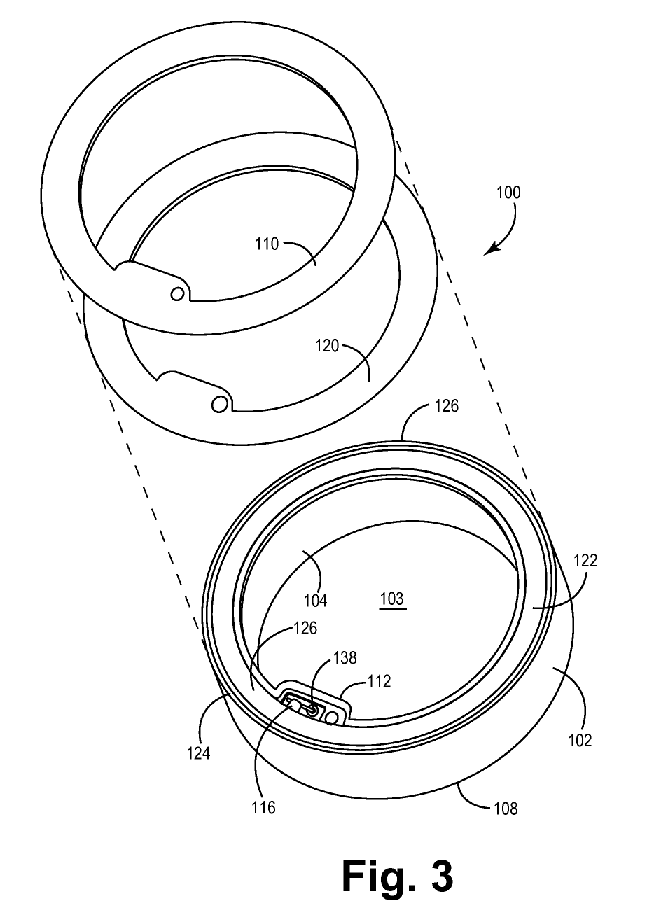

FIG. 1 is an isometric view of a torroidal battery 100 in accordance with a first embodiment of the present invention. Torroidal battery 100 comprises a generally torroidal or doughnut-shaped housing 102 (e.g., titanium, aluminum, stainless steel, etc.) having a central opening 103 therethrough. Torroidal housing 102 comprises a substantially circular inner wall 104, a substantially circular outer wall 106, and a housing cover 110. Housing cover 110 is fixedly coupled to the upper edges of walls 104 and 106 by, for example, laser welding. A protrusion or shelf 112 extends from a section of inner wall 104 into central opening 103. A fill port 114 is provided through shelf 112 to allow the introduction of an electrolytic fluid into torroidal housing 102. The electrolytic fluid enables ionic communication between electrodes disposed within housing 102, which are described in greater detail herein below. After battery 100 has been filled with an electrolytic fluid, a cover (not shown) may be inserted over fill port 114 and fixedly coupled (e.g., laser welded) to housing cover 110 to ensure that electrolytic fluid does not escape from battery 100. As shown in FIG. 2, an isometric view of the underside of shelf 112, shelf 112 also includes an aperture 118 therethrough to accommodate a first, exposed end of a lead 116 (e.g., a niobium terminal pin). This end of lead 116 may be electrically coupled to one or more electrical components disposed within central opening 103. The other end of lead 116, discussed below in conjunction with FIG. 3, may be electrically couple (e.g., welded) to an electrode disposed within torroidal housing 102.

FIG. 3 is a partially exploded view of torroidal battery 100. Housing cover 110 and an insulative cover 120 (e.g., polypropylene) have been removed from battery 100 to expose an electrode assembly 122. Electrode assembly 122 resides within an inner annular cavity 124 provided within torroidal housing 102 between inner wall 104 and outer wall 106. An insulative body 126 (e.g., a polypropylene insert) is also disposed within inner annular cavity 124 intermediate electrode assembly 122 and torroidal housing 102. Insulative body 126 electrically isolates electrode assembly 122 from torroidal housing 102 to prevent the shorting of battery 100. The second end of lead 116 is also exposed in FIG. 3. This end of lead 116 is generally bent or J-shaped and emerges within shelf 112. Lead 116 is secured relative to torroidal housing 102, and electrically isolated therefrom, by a feedthrough assembly 138 that is fixedly coupled (e.g., welded) to shelf 112. Feedthrough assembly 138 may comprise, for example, a metal ferrule (e.g., titanium) having an insulative structure (e.g., glass) disposed therein. The insulative structure secures and insulates lead 116 within the ferrule of feedthrough assembly 138. The insulative structure also forms a hermetic seal within the ferrule.

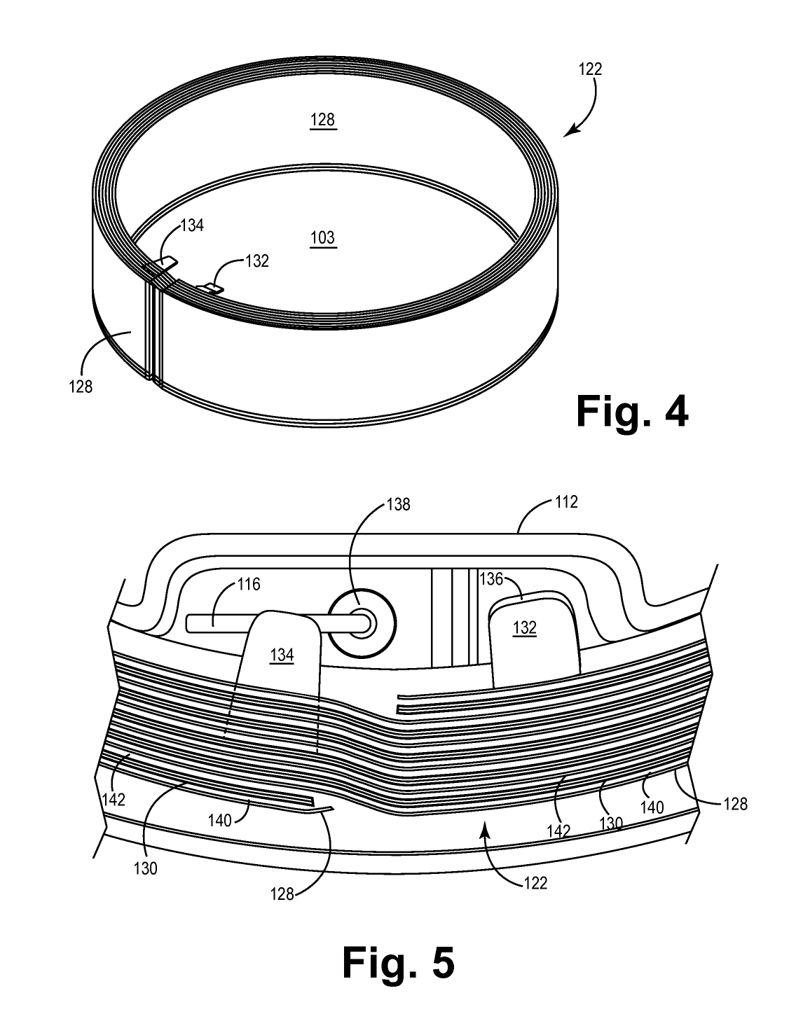

FIG. 4 illustrates electrode assembly 122 prior to insertion into torroidal housing 102. Electrode assembly 122 comprises a first electrode 128 (e.g., an anode) and a second electrode 130 (e.g., a cathode). Electrodes 128 and 130 are initially produced as relatively long strips of foil that are coiled together as described below to form the annular body of electrode assembly 122. Electrodes 128 and 130 may each comprise a body of active material (e.g., an anode-type metal, such as lithium; or a cathode-type mix, such as silver vanadium oxide powder) having a current collector disposed therein. The current collector may take of the form of, for example, a flattened metal plate (e.g., titanium) having a plurality (e.g., a grid) of apertures therethrough. Electrodes 128 and 130 are each provided with a lead extending therefrom that may serve as an electrical contact. For example, electrodes 128 and 130 may be provided with inner tabs 132 and 134, respectively. If electrode 128 or electrode 130 includes a current collector, tab 132 or 134 may comprise an exposed portion of an elongated stem extending from the body of the current collector.

FIG. 5 is a top view of shelf 112 and a section of electrode assembly 122. Here, it may be seen that electrode assembly 122 includes a separator material disposed between electrodes 128 and 130 to preclude physical contact and electrical shorting between the electrodes. The separator material is porous so as to permit the passage of ions and may comprise, for example, a polymeric film (e.g., polypropylene, polyethylene, etc.). During the coiling process, a first layer of separator material 140 is placed over electrode 128, electrode 130 is placed over layer 140, and then a second layer of electrode material 142 is placed over electrode 130. The resulting laminate, which comprises electrodes 128 and 130 and separator material layers 140 and 142, is then coiled around a mandrill (e.g., a tube or disc) having an outer diameter equivalent to, or slightly larger than, the outer diameter of inner wall 104 (FIGS. 1-3). The mandrill is subsequently removed, and the coiled electrode assembly 122 is inserted into to inner annular cavity 124 of torroidal housing 102. Significantly, assembly of torroidal battery 100 does not require the tight coiling of electrode assembly 122. Thus, relative to conventional cylindrical battery designs, the inventive torroidal battery design decreases the likelihood of damaging electrodes 128 and 130 during manufacture and facilitates insertion of electrode assembly 122 into housing 102. Additionally, during manufacture of battery 100, the inner annular surface of electrode assembly 122 is exposed as shown in FIG. 4. This facilitates the inspection of electrode assembly 122 prior to insertion, especially inspection of the inner annular surface of electrode assembly 122.

As stated above, tabs 132 and 134 provide electrical contacts for electrodes 128 and 130, respectively. Tab 132 may be welded to, for example, a portion of shelf 112 to electrically couple electrode 128 to torroidal housing 102. To permit tab 132 to be so coupled, an aperture 136 is provided through a portion of insulative body 126 overlapping shelf 112. In contrast, tab 134 may be welded to the second end of lead 116. This electrically couples electrode 130 to lead 116 and, therefore, to any circuitry to which the first end of lead 116 (FIG. 2) may be coupled. Notably, the positioning of tabs 132 and 134, and the general torroidal design of battery 100, provides an area in which welding may be performed without a substantial risk of damage to other components of battery 100 or to other components of an IMD in which battery 100 is deployed.

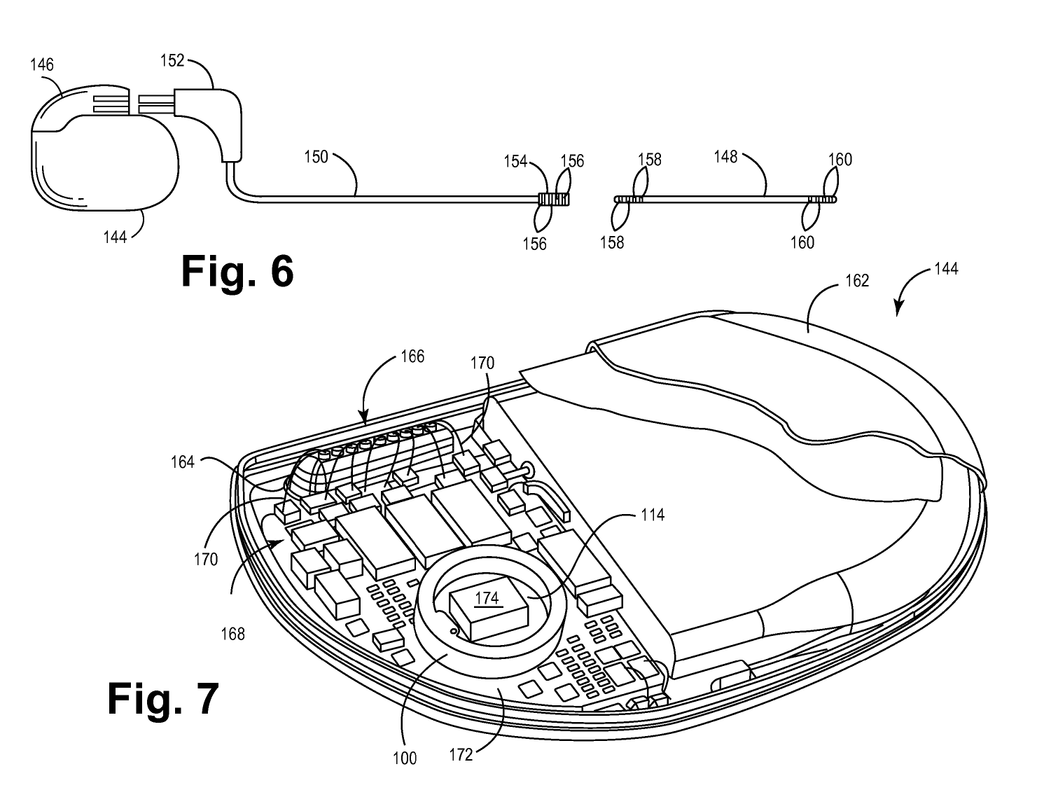

Due to its volumetric efficiency and other associated advantages described herein, torroidal battery 100 is ideal for implementation within an IMD. FIG. 6 is an exploded view of an implantable medical device 143 including a pulse generator 144 in which torroidal battery 100 may be employed. Pulse generator 144 includes a connector block 146, which is coupled to a lead 148 by way of an extension 150. The proximal portion of extension 150 comprises a connector 152 configured to be received or plugged into connector block 146, and the distal end of extension 150 likewise comprises a connector 154 including internal electrical contacts 156. Electrical contacts 156 are configured to receive the proximal end of lead 148 having a plurality of electrical contacts 158 disposed thereon. The distal end of lead 148 includes distal electrodes 160, which may deliver therapy (e.g., defibrillating electrical pulses) to one or more target areas or sense signals (e.g., cardiac signals) generated within a patient's body.

FIG. 7 is an isometric cutaway view of pulse generator 144 (FIG. 6) illustrating one manner in which torroidal battery 100 may be deployed within an implantable medical device. Pulse generator 144 comprises a canister 162 (e.g. titanium or other biocompatible material) having an aperture 164 therein, which accommodates a multipolar feedthrough assembly 166. Circuitry 168 is provided within battery 100 and resides upon a printed circuit board 172. Circuitry 168 is coupled to each of the terminal pins of feedthrough assembly 166 via a plurality of connective wires 170 (e.g., gold). Torroidal battery 100 may also be mounted on circuit board 172 and coupled to circuitry 168 via one or more connective wires. In particular, torroidal battery 100 may be electrically coupled to one or more components of circuitry (e.g., a capacitor, a drug reservoir, etc.) disposed within central opening 114. As shown in FIG. 7, for example, an integrated chip 174 may be disposed within opening 114. Chip 174 may be coupled to battery 100 by way of a connective wire having a first end bonded to an external contact provided on chip 174 and a second end bonded to the exposed end of lead 116 (FIG. 2). Battery 100 may thus provide power to pulse generator 144 thereby enabling IMD 143 to deliver therapy to treatment sites within a patient's body. Due to its generally torroidal shape, and by permitting components of circuitry 168 to be disposed within central opening 114, torroidal battery 100 provides a significant space-saving advantage over other conventional battery designs (e.g., cylindrical battery designs).

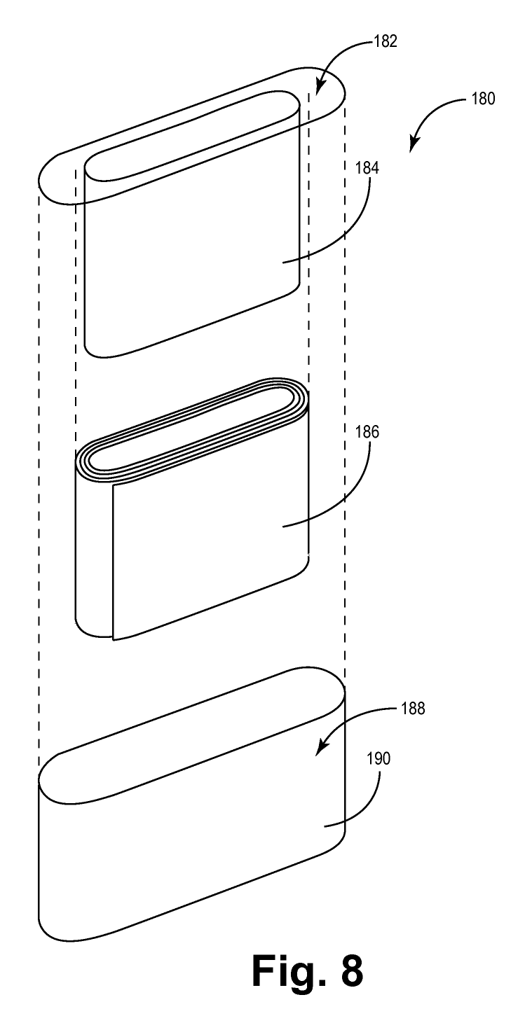

As exemplary battery 100 has been described and shown herein as having a torroidal shape, it should be made clear that the term “torroid” is used in a broad and generalized sense. The inventive battery may assume other shapes similar to a torroid and still be considered torroidal for purposes of this application. This generally includes, but is not limited to, shapes having a rounded (e.g., a generally rounded polygonal) or elliptical inner wall defining a central opening through the battery's canister. To further illustrate this point, FIG. 8 provides an isometric exploded view of a torroidal battery 180. Battery 180 comprises a first housing piece 182 including an inner wall 184, a coiled electrode assembly 186, and a second housing piece 188 having an outer wall 190. Unlike battery 100 (FIGS. 1-5 and 7), inner wall 184 and outer wall 190 have a generally elliptical shape. Thus, battery 180 may be preferable to battery 100 if, for example, the torroidal battery is to be disposed within a generally rectangular space on a printed circuit board.

Importantly, battery 180 differs from battery 100 in another manner; i.e., the inner wall of battery 180 (i.e., inner wall 184 of housing piece 182) is exposed and easily accessed prior to assembly. This permits inner wall 184 to serve as a mandrill around which electrode assembly 186 may be coiled. After coiling electrode assembly 186 around inner wall 184, housing piece 182 and electrode assembly 186 may be lowered into housing piece 188, and piece 182 may be welded to piece 188. Thus, the design of torroidal battery 180 simplifies the coiling and insertion process by rendering unnecessary the additional step of coiling electrode assembly 186 around, and removing assembly 186 from, a separate mandrill.

In view of the above, it should be appreciated that a torroidal battery has been provided for use in an IMD that occupies a relatively small volume of space and that overcomes many of the limitations associated with conventional cylindrical battery designs. Although the invention has been described with reference to a specific embodiment in the foregoing specification, it should be appreciated that various modifications and changes can be made without departing from the scope of the invention as set forth in the appended claims. Accordingly, the specification and figures should be regarded as illustrative rather than restrictive, and all such modifications are intended to be included within the scope of the present invention.

What is claimed is:

1. A torroidal battery for use in an implantable medical device, comprising:
a torroidal canister having a central opening therethrough;
an electrode assembly disposed within said canister; and
an insulative body disposed between said torroidal canister and said electrode assembly.

2. A torroidal battery according to claim 1 wherein said electrode assembly is coiled.

3. A torroidal battery according to claim 1 wherein said electrode assembly comprises a first electrode, a second electrode, and a separator material disposed between said first electrode and said second electrode.

4. A torroidal battery according to claim 3 wherein said first electrode includes a first tab extending therefrom, said first tab electrically coupled to a portion of said torroidal canister.

5. A torroidal battery according to claim 4 wherein said portion comprises a shelf extending from said torroidal canister into the central opening.

6. A torroidal battery according to claim 1 further comprising a feedthrough assembly fixedly coupled to said torroidal canister, said feedthrough assembly including a lead having a first end electrically coupled to said electrode assembly.

7. A torroidal battery according to claim 6 wherein said lead has a second end, said second end residing within the central opening.

8. A torroidal battery according to claim 1 further comprising a fill port through said torroidal canister, said fill port configured to permit the introduction of electrolytic fluid into said torroidal canister.

9. A torroidal battery according to claim 1 wherein said canister includes a mandrill around which said electrode assembly is disposed.

10. An implantable medical device, comprising:
a housing;
circuitry disposed within said housing; and
a torroidal battery disposed within said housing and coupled to said circuitry.

11. An implantable medical device according to claim 10 wherein said torroidal battery comprises:
a torroidal canister having a central opening therethrough;
an electrode assembly disposed within said canister; and
an insulative body disposed between said canister and said electrode assembly.

12. An implantable medical device according to claim 11 further comprising a feedthrough assembly through said torroidal canister, said feedthrough assembly including a lead having a first end coupled to said circuitry and a second end coupled to said electrode assembly.

13. An implantable medical device according to claim 10 further comprising a circuit board, said torroidal battery and at least a portion of said circuitry mounted on said circuit board.

14. An implantable medical device according to claim 13 wherein a portion of said circuitry is disposed within the central opening.

15. An implantable medical device according to claim 14 wherein said first end is exposed through the central opening and wherein said lead is coupled to said portion of said circuitry.

16. An implantable medical device according to claim 12 wherein said torroidal canister comprises an inner annular portion proximate said central opening and a shelf extending therefrom, said feedthrough assembly disposed through said shelf.

17. An implantable medical device according to claim 16 wherein said electrode assembly comprises a first coiled electrode electrically coupled to said lead and a second coiled electrode electrically coupled to said shelf.

18. An implantable medical device, comprising:
a housing;
circuitry disposed within said housing; and
a torroidal battery disposed within said housing and coupled to said circuitry, said torroidal battery comprising:
a torroidal canister having an inner wall, an outer wall, and an inner annular cavity;
a coiled electrode assembly disposed within said torroidal canister and around said inner wall;
an insulative body disposed between said torroidal canister and said coiled electrode assembly; and
a feedthrough assembly through said torroidal canister and said insulative body, said feedthrough assembly having a lead therethrough coupled to said coiled electrode assembly.

19. An implantable medical device according to claim 18 wherein said inner wall defines a central opening through said torroidal canister, and wherein a portion of said circuitry resides within the central opening.

20. An implantable medical device according to claim 19 wherein said lead is disposed through said inner wall and coupled to said portion of said circuitry.
